(19) United States
(12) Patent Application Publication
(10) Pub. No.: US 2024/0153036 A1
(43) Pub. Date: May 9, 2024

(54) MEDICAL IMAGE PROCESSING SYSTEM, MEDICAL IMAGE PROCESSING METHOD, AND PROGRAM

(71) Applicant: SONY GROUP CORPORATION, TOKYO (JP)

(72) Inventors: MASAHITO YAMANE, TOKYO (JP); YUKI SUGIE, TOKYO (JP); TSUNEO HAYASHI, TOKYO (JP)

(21) Appl. No.: 18/550,541

(22) PCT Filed: Jan. 20, 2022
(86) PCT No.: PCT/JP2022/001916

§ 371 Date: Sep. 14, 2023

(30) Foreign Application Priority Data
Mar. 25, 2021 (JP) 2021-050944

(51) Int. Cl. G06T 5/00; G06T 7/11; G16H 30/40

(52) U.S. Cl. CPC G06T 5/00; G06T 7/11; G16H 30/40; G06T 2207/20021; G06T 2207/30004

(57) ABSTRACT

The present disclosure relates to a medical image processing system that is capable of implementing low-latency image processing, a medical image processing method, and a program.

An image processing unit executes, at each predetermined processing timing, image processing on each of a plurality of medical images input asynchronously in units of strip images obtained by dividing each of the medical images into a plurality of pieces. The present disclosure can be applied to the medical image processing system.

### TECHNICAL FIELD

The present disclosure relates to a medical image processing system, a medical image processing method, and a program, and more particularly, to a medical image processing system that implement low-latency image processing, a medical image processing method, and a program.

### BACKGROUND ART

In an operation using a medical imaging device (image transmission device) such as an endoscope or a video microscope, images subjected to various image processing are output such that a more detailed procedure can be performed. Such image processing is required to be executed with a low latency so as not to interfere with a procedure or a manipulation.

On the other hand, in a medical facility such as an operating room or a hospital, medical images output from various image transmission devices are displayed by an image reception device such as a monitor or recorded in an external storage. Among them, it is general that the image transmission device and the image reception device are not directly connected but are connected via a low-latency Internet Protocol (IP) network in the medical facility. In this IP network, one image reception device can receive and display a medical image from the image transmission device by synchronizing with one image transmission device.

Patent Document 1 discloses a synchronization control system that, in an IP network, receives setting information and a time code from an imaging device, provides a latency on the basis of on the setting information, and synchronizes with a display device in a network to which a transmission source belongs.

Furthermore, Patent Document 2 discloses an operation system capable of displaying an image captured with a low latency in a state close to real time.

### CITATION LIST

### Patent Document

Patent Document 1: Japanese Patent Application Laid-Open No. 2020-5063 A
Patent Document 2: WO 2015/163171 A

### SUMMARY OF THE INVENTION

### Problems to be Solved by the Invention

The technology of Patent Document 1 controls a latency of an asynchronous image signal, and cannot execute image processing with a low latency on the image signal itself. Furthermore, the technology of Patent Document 2 copes with only a single image signal, and cannot cope with a plurality of image signals at the same time. That is, the image processing on each of a plurality of medical images input asynchronously cannot be executed with a low latency.

The present disclosure has been made in view of such a situation, and implements low-latency image processing.

### Solutions to Problems

According to an aspect of the present disclosure, there is provided a medical image processing system including an image processing unit configured to execute, at each predetermined processing timing, image processing on each of a plurality of medical images input asynchronously in units of strip images obtained by dividing each of the medical images into a plurality of pieces.

According to another aspect of the present disclosure, there is provided a medical image processing method including causing a medical image processing system to execute, at each predetermined processing timing, image processing on each of a plurality of medical images input asynchronously in units of strip images obtained by dividing each of the medical images into a plurality of pieces.

According to still another aspect of the present disclosure, there is provided a program causing a computer to execute, at each predetermined processing timing, image processing on each of a plurality of medical images input asynchronously in units of strip images obtained by dividing each of the medical images into a plurality of pieces.

In the aspects of the present disclosure, at each predetermined processing timing, the image processing on each of a plurality of medical images input asynchronously is executed in units of strip images obtained by dividing each of the medical images into a plurality of pieces.

### BRIEF DESCRIPTION OF DRAWINGS

FIG. 1 is a block diagram illustrating a configuration example of a medical image processing system of the related art.

FIG. 2 is a block diagram illustrating a configuration example of a medical image processing system to which the technology according to the present disclosure can be applied.

FIG. 3 is a block diagram illustrating a hardware configuration example of an image processing server.

FIG. 4 is a stack diagram illustrating hardware and software of an image processing server.

FIG. 5 is a diagram illustrating a functional configuration example of an image processing server.

FIG. 6 is a diagram for explaining a strip image.

FIG. 7 is a flowchart for explaining a flow of image processing.

FIG. 8 is a diagram illustrating a specific example of a processing timing of image processing.

FIG. 9 is a diagram illustrating a configuration example of a computer.

### MODE FOR CARRYING OUT THE INVENTION

A mode for carrying out the present disclosure (hereinafter, referred to as an embodiment) will be described below. Note that the description will be given in the following order.
1. Network Configuration of Related Art
2. Technical Background and Problems in Recent Years
3. Configuration of Image Processing Server
4. Flow of Image Processing
5. Configuration Example of Computer

<1. Network Configuration of Related Art>

In an operation using an image transmission device such as an endoscope or a video microscope, an image subjected to various types of image processing such as noise removal, distortion correction, improvement of a sense of resolution, improvement of a sense of gradation, color reproduction, color enhancement, and digital zoom is output such that a more detailed procedure can be performed.

Such image processing is required to be executed with a low latency on a large amount of data having high resolution such as 4 K and a high frame rate such as 60 fps so as not to interfere with a procedure or a manipulation. Furthermore, in a case where fluctuation occurs in the processing time and the prescribed frame rate cannot be satisfied, a phenomenon that the image movement is awkward occurs, and thus there is a possibility that the procedure is interfered. Therefore, it is required to implement image processing (real-time image processing) that satisfies such performance requirements (real-time property).

On the other hand, in a medical facility such as an operating room or a hospital, medical images output from various image transmission devices are displayed by an image reception device such as a monitor or recorded in an external storage. Among them, it is general that the image transmission device and the image reception device are not directly connected but are connected via a low-latency network in the medical facility. Such a network is referred to as an IP network, a video over IP (VoIP) network, or the like. In the IP network, it is possible to display, on an arbitrary monitor, medical images from various devices used in the operation, such as an endoscope, an ultrasound diagnosis device, and a biological information monitor, and to switch the display.

In general, the image transmission device or the image reception device does not include a connection terminal for direct connection to the IP network. Therefore, an IP converter for mutually converting an input/output signal and an IP signal of the image transmission device or the image reception device is required.

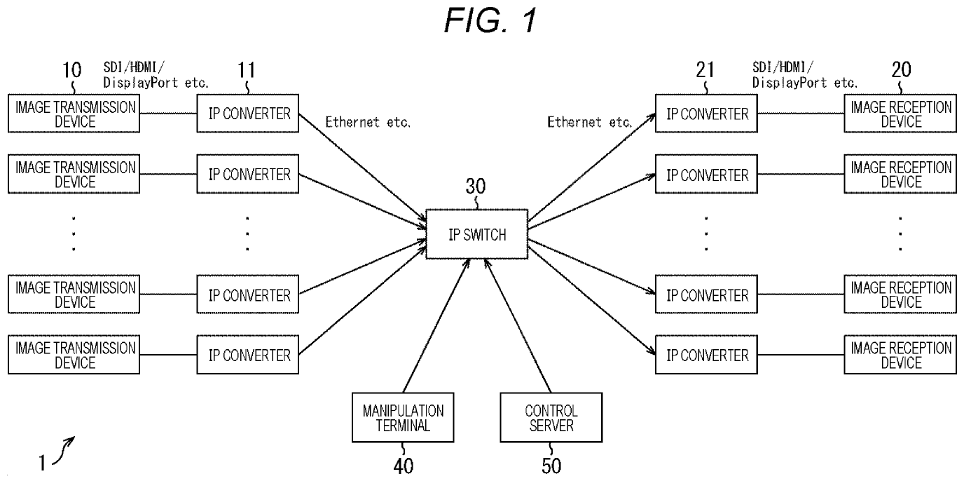

FIG. 1 is a block diagram illustrating a configuration example of a medical image processing system of the related art.

A medical image processing system 1 of FIG. 1 includes an image transmission device 10, an IP converter 11, an image reception device 20, an IP converter 21, an IP switch 30, a manipulation terminal 40, and a control server 50.

In the medical image processing system 1, a plurality of the image transmission devices 10 is provided, and the IP converters 11 corresponding to the number of image transmission devices 10 are also provided. Similarly, a plurality of the image reception devices 20 is provided, and the IP converters 21 corresponding to the number of image reception devices 20 are also provided.

Each of the image transmission devices 10 is connected to the IP switch 30 via the IP converter 11. Furthermore, each of the image reception devices 20 is connected to the IP switch 30 via the IP converter 21. The image transmission device 10 and the image reception device 20 have interfaces such as a serial digital interface (SDI), a high-definition multimedia interface (HDMI) (registered trademark), and a display port.

The IP converter 11 converts a medical image (image signal) from the image transmission device 10 into an IP signal and outputs the IP signal to the IP switch 30. Furthermore, the IP converter 21 converts the IP signal from the IP switch 30 into an image signal and outputs the image signal to the image reception device 20.

The IP switch 30 controls input and output of an image signal to and from the connected device on the basis of the control of the control server 50. Specifically, the IP switch 30 controls high-speed transfer of the image signal between the image transmission device 10 and the image reception device 20, which are disposed on the IP network.

The manipulation terminal 40 is configured as a personal computer (PC), a tablet terminal, a smartphone, or the like manipulated by a user. The manipulation terminal 40 receives selection of the image reception device 20 as an output destination of the medical image output from the image transmission device 10 on the basis of the user's manipulation.

The control server 50 sets (performs routing on) the image reception device 20 as an output destination of the medical image output from the image transmission device 10 by controlling the IP switch 30 on the basis of the user's manipulation on the manipulation terminal 40.

In the IP network configuring the medical image processing system, one image reception device 20 can receive and display the medical image from the image transmission device 10 by synchronizing with one image transmission device 10.

<2. Technical Background and Problems in Recent Years>

(Background in Recent Years)

In recent years, a medical application that supports a procedure by performing not only image processing but also high-load arithmetic processing such as image recognition by artificial intelligence (AI) has been put into practical use.

In order to realize such a medical application, it is conceivable to dispose a general-purpose server on an IP network in addition to introducing a new medical imaging device (image transmission device) including a high-load arithmetic processing mechanism. In recent years, performance of a graphics processing unit (GPU) card for a general-purpose server has been improved. From this, it is conceivable that the general-purpose server can provide a function equivalent to that of the above-described medical application by acquiring a medical image from the image transmission device, performing image processing with software, and transmitting the medical image to the image reception device. Hereinafter, the general-purpose server having such a function is referred to as an image processing server.

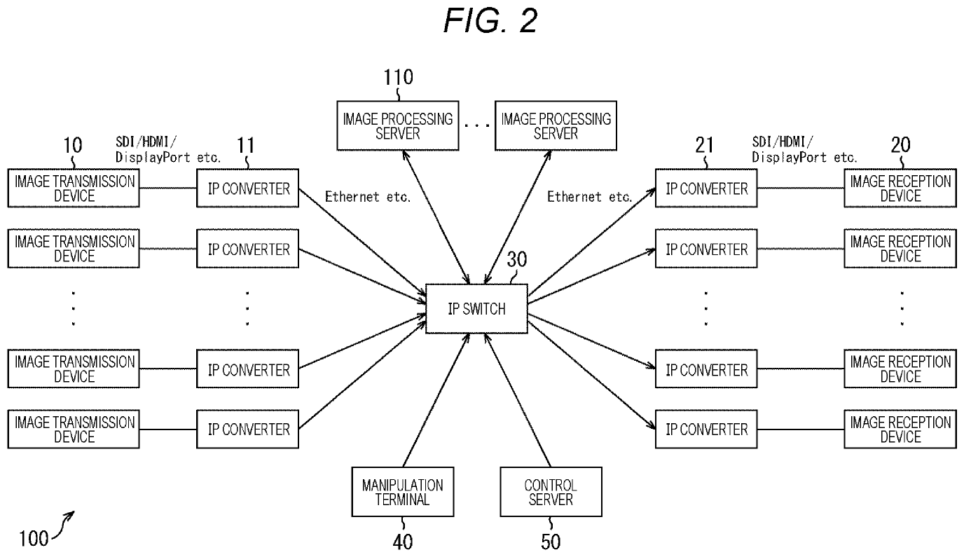

FIG. 2 is a block diagram illustrating a configuration example of the medical image processing system to which the technology according to the present disclosure can be applied.

A medical image processing system 100 of FIG. 2 is configured to include an image processing server 110 in addition to the same configuration as that of FIG. 1.

The image processing server 110 is connected to the IP switch 30, acquires a medical image from the image transmission device 10 via the IP converter 11, and performs image processing with software. The image processing server 110 transmits the medical image subjected to the image processing to the image reception device 20 via the IP converter 21. The routing between the image transmission device 10 and the image reception device 20 is performed by the control server 50 similarly to the medical image processing system 1 in FIG. 1.

One image processing server 110 can receive medical images from a plurality of the image transmission devices 10, perform image processing in parallel, and transmit the processed medical images to a plurality of the image reception devices 20. In the medical image processing system 100, there may be a plurality of the image processing servers 110.

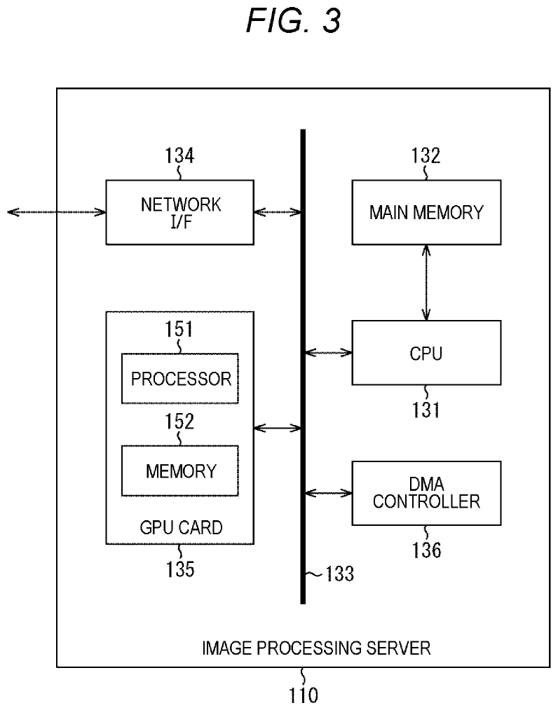

FIG. 3 is a block diagram illustrating a hardware configuration example of the image processing server 110.

The image processing server 110 includes a central processing unit (CPU) 131, a main memory 132, a bus 133, a network interface (I/F) 134, a GPU card 135, and a direct memory access (DMA) controller 136.

The CPU 131 controls the entire operation of the image processing server 110.

The main memory 132 temporarily stores a medical image (image data) from the image transmission device 10. The image data temporarily stored in the main memory 132 is subjected to image processing in the GPU card 135, and is stored again in the main memory 132. The Image data subjected to the image processing, which is stored in the main memory 132, is transmitted to the image reception device 20.

The network I/F 134 receives the image data supplied from the image transmission device 10, and supplies the image data to the main memory 132 or the GPU card 135 via the bus 133. Furthermore, the network I/F 134 transmits the image data subjected to the image processing, which is supplied from the main memory 132 or the GPU card 135, to the image reception device 20 via the bus 133.

The GPU card 135 includes a processor 151 and a memory (GPU memory) 152. The GPU card 135 temporarily stores the image data supplied from the main memory 132 or the network I/F 134 via the bus 133 in the memory 152 under the management of the DMA controller 136. The processor 151 performs predetermined image processing while sequentially reading the image data stored in the memory 152. Furthermore, the processor 151 buffers the processing result in the memory 152 as necessary, and outputs the processing result to the main memory 132 or the network I/F 134 via the bus 133.

The DMA controller 136 directly transfers (performs DMA transfer of) data to the network I/F 134, the main memory 132, and the GPU card 135 via the bus 133 without being managed by the CPU 131. Specifically, the DMA controller 136 controls a transfer source and transfer destination, and a transfer timing in the DMA transfer.

Therefore, a plurality of pieces of asynchronous image data transmitted from a plurality of the image transmission devices 10 is received by the network I/F 134 and temporarily relayed to the main memory 132 or directly transferred to the memory 152 of the GPU card 135. The image data transferred to the memory 152 is subjected to image processing by the processor 151, and the processing result is stored in the memory 152 again. The image data subjected to the image processing, which is stored in the memory 152, is temporarily relayed to the main memory 132 or directly transferred to the network I/F 134, and transmitted to a plurality of the image reception devices 20.

Note that in the image processing server 110, a plurality of the CPUs 131, a plurality of the network I/Fs 134, and a plurality of the GPU cards 135 may be provided. Furthermore, the DMA controller 136 may be provided inside the CPU 131.

(Problems)

In the image processing server 110 as illustrated in FIG. 3, in order to implement a function equivalent to that of the above-described medical application, it is required to perform image processing with a low latency on a plurality of medical images input asynchronously so as not to interfere with a procedure.

However, in parallel processing with software on a general server, in a case where an application for executing image processing on each of a plurality of pieces of image data is activated, access to the network I/F 134 and the GPU card 135 is performed at each individual timing. The access arbitration is performed by an operating system (OS) or a device driver. At this time, since the OS and the device driver perform access control focusing on the overall throughput, it is difficult to ensure a low latency. Furthermore, since the application also accesses the GPU card 135 at individual timing, overhead may be increased.

Therefore, in the image processing server 110 to which the technology of the present disclosure is applied, it is possible to execute real-time image processing with a low latency in parallel on each of a plurality of the medical images input asynchronously.

<3. Configuration of Image Processing Server>

A configuration of the image processing server 110 to which the technology of the present disclosure is applied will be described. Note that the hardware configuration of the image processing server 110 is as described with reference to FIG. 3.

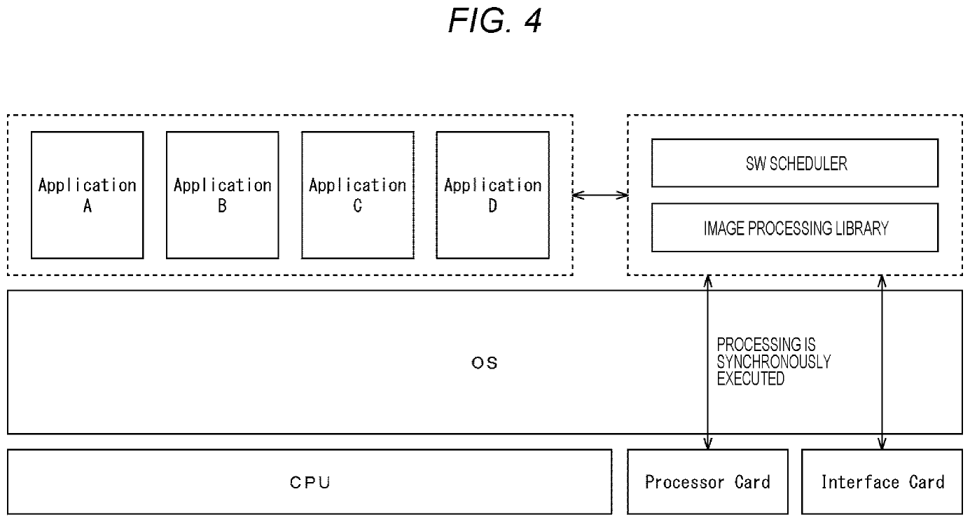

FIG. 4 is a stack diagram illustrating the hardware and software of the image processing server 110.

The image processing server 110 includes three layers of a hardware layer, an OS layer, and an application layer.

The lower hardware layer includes various types of hardware such as a CPU (corresponding to the CPU 131), a processor card (corresponding to the GPU card 135), and an interface card (corresponding to the network I/F 134).

In the intermediate OS layer, there is an OS that operates on the hardware layer.

The upper application layer includes various applications operating on the OS layer.

In the example of FIG. 4, four applications A to D and a software (SW) scheduler operate in the application layer. The image processing performed on each of a plurality of the medical images transmitted from a plurality of the image transmission devices 10 is defined by the applications A to D. Each actual image processing is executed by the SW scheduler while the SW scheduler refers to an image processing library. The SW scheduler is implemented by the processor 151 of the GPU card 135.

While the medical images from a plurality of the image transmission devices 10 are asynchronously input to the image processing server 110, the image processing performed on each of the medical images is synchronously executed at a predetermined processing timing by the SW scheduler.

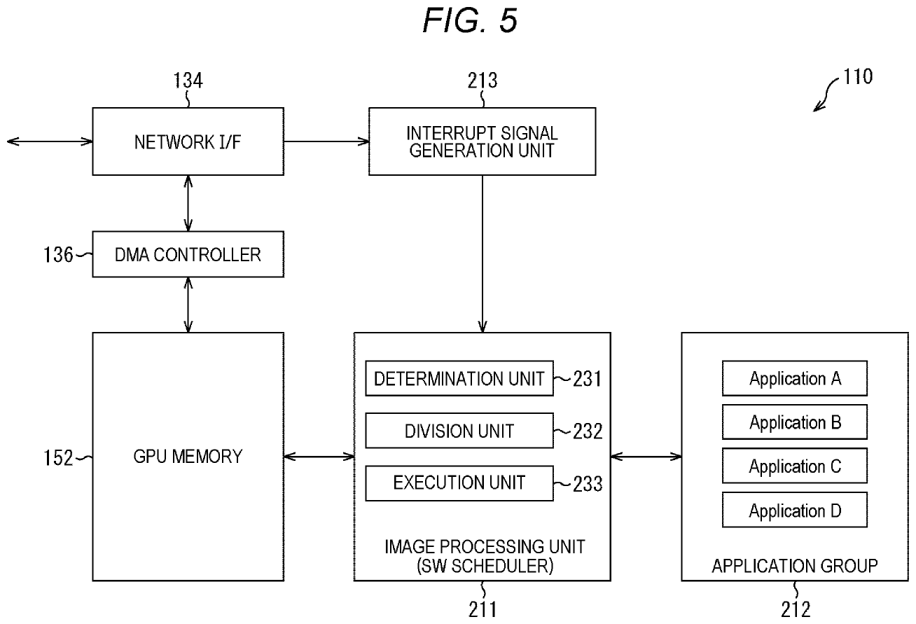

FIG. 5 is a block diagram illustrating a functional configuration example of the image processing server 110.

The image processing server 110 illustrated in FIG. 5 includes a network I/F 134, a DMA controller 136, a GPU memory 152, an image processing unit 211, an application group 212, and an interrupt signal generation unit 213. Note that in the image processing server 110 of FIG. 5, the same components as those of the image processing server 110 of FIG. 3 are denoted by the same reference numerals, and the description thereof will be appropriately omitted.

The image processing unit 211 corresponds to the SW scheduler of FIG. 4, and is implemented by the processor 151 of the GPU card 135. The image processing unit 211 performs image processing defined by the application included in the application group 212 on each of the medical images transferred to the GPU memory 152.

The application included in the application group 212 is prepared (installed) for each medical image to be subjected to image processing.

The interrupt signal generation unit 213 may also be implemented by the processor 151 of the GPU card 135 and configured as a part of the SW scheduler.

The interrupt signal generation unit 213 generates an interrupt signal for driving the image processing unit 211. Specifically, the interrupt signal generation unit 213 generates a synchronization signal having a frequency equal to or higher than a frequency of a vertical synchronization signal of all the medical images that may be input to the image processing server 110. Then, the interrupt signal generation unit 213 generates an interrupt signal by multiplying the synchronization signal by a predetermined multiplication number.

The frequencies of the vertical synchronization signals of all the medical images that may be input to the image processing server 110 may be manually set in the manipulation terminal 40, or may be provided in notification from the IP converter 11 to the image processing server 110 via the control server 50 or directly.

The synchronization signal and interrupt signal generated by the interrupt signal generation unit 213 may be clocks such as a read time stamp counter (RDTSC) included in the CPU 131 (FIG. 3). Furthermore, the synchronization signal and the interrupt signal may be clocks generated from the network I/F 134 or a dedicated PCI-E (Express) board.

The image processing unit 211 includes a determination unit 231, a division unit 232, and an execution unit 233.

On the basis of the interrupt signal from the interrupt signal generation unit 213, the determination unit 231 determines whether or not it is a processing timing to execute image processing on each medical image.

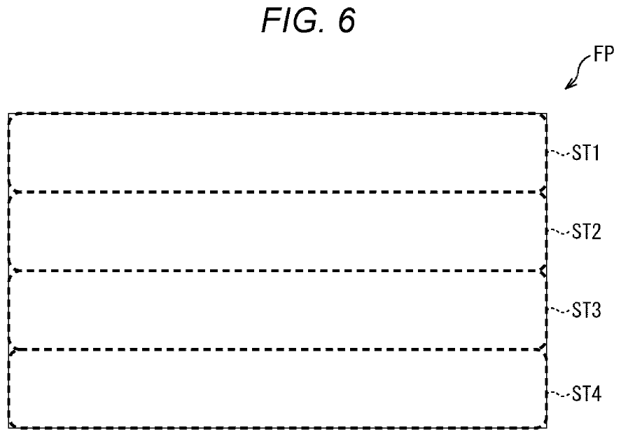

The division unit 232 horizontally divides each of frames constituting each medical image transferred to the GPU memory 152 into a plurality of frames. For example, the division unit 232 divides a frame image FP illustrated in FIG. 6 into four regions in a horizontal direction. Images corresponding to four regions ST1, ST2, ST3, and ST4 obtained by dividing the frame image FP, which are indicated by broken lines in the drawing, are referred to as strip images.

Note that the multiplication number for multiplying the synchronization signal when the interrupt signal generation unit 213 generates the interrupt signal is a division number of the strip image. That is, the strip image can also be referred to as an execution unit of the image processing on the medical image.

The execution unit 233 executes the image processing on each medical image in units of divided images at each processing timing described above.

With the above-described configuration, the image processing unit 211 can execute image processing on each medical image in units of strip images obtained by dividing each of the medical images into a plurality of pieces at each processing timing at which the interrupt signal is supplied from the interrupt signal generation unit 213.

<4. Flow of Image Processing>

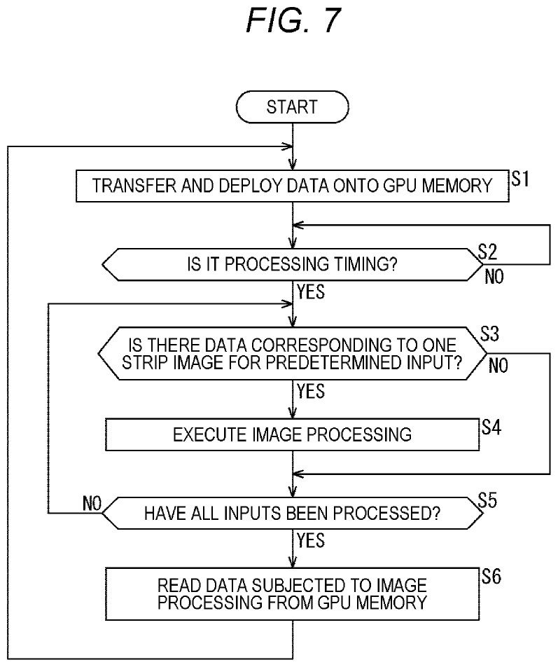

Here, a flow of the image processing by the image processing server 110 in FIG. 5 is described with reference to a flowchart in FIG. 7.

Here, a plurality of the medical images is asynchronously input from a plurality of the image transmission devices 10 to the image processing server 110 via the IP converter 11. The image processing server 110 performs image processing on each of a plurality of the medical images, and outputs the medical image to each of the image reception devices 20 as an output destination via the IP converter 21.

In step S1, the DMA controller 136 transfers and deploys the medical image (image data) from the image transmission device 10, which is received by the network I/F 134, onto the GPU memory 152 in raster order.

In step S2, the determination unit 231 (SW scheduler) of the image processing unit 211 determines whether or not it is a processing timing to execute image processing on the basis of the interrupt signal from the interrupt signal generation unit 213.

Step S2 is repeated until it is determined that the timing is the processing timing, that is, until the interrupt signal is supplied from the interrupt signal generation unit 213. Then, when the interrupt signal is supplied from the interrupt signal generation unit 213 and it is determined that the timing is the processing timing, the processing proceeds to step S3.

In step S3, the division unit 232 (SW scheduler) of the image processing unit 211 determines whether or not there is image data corresponding to one strip image (deployment is completed) on the GPU memory 152 for a predetermined input among a plurality of inputs (medical images).

When it is determined in step S3 that there is image data corresponding to one strip image for the input, the processing proceeds to step S4, and the execution unit 233 (SW scheduler) of the image processing unit 211 executes image processing corresponding to the input on the image data corresponding to one strip image.

Note that the image data may be deployed to different region on the GPU memory 152 for each strip image.

On the other hand, when it is determined in step S3 that there is no image data corresponding to one strip image for the input (deployment is not completed), step S4 is skipped.

Thereafter, in step S5, the division unit 232 (SW scheduler) of the image processing unit 211 determines whether or not all the inputs (medical images) have been processed (whether or not steps S3 and S4 have been executed).

In step S5, when it is determined that all the inputs are not processed, the processing returns to step S3, and steps S3 and S4 are repeated. On the other hand, when it is determined that all the inputs have been processed, the processing proceeds to step S6.

In step S6, the DMA controller 136 reads the image data subjected to the image processing in units of strip images in raster order from the GPU memory 152, and transfers the read image data to the network I/F 134. The image data transferred to the network I/F 134 is output to the image reception device 20 corresponding to the image transmission device 10 to which the image data not subjected to the image processing is input.

In the above-described processing, it is possible to implement low-latency image processing until the medical image is output to the image reception device 20 on the IP network after the medical image from the image transmission device 10 is input.

The above-described processing is repeated while a plurality of the medical images is asynchronously input to the image processing server 110. Under the circumstances, the image processing server 110 (DMA controller 136) delays a timing to output the image data subjected to the image processing from the GPU memory 152 to the network I/F 134 according to the division number of the strip image with respect to a timing to input the image data not subjected to the image processing from the network I/F 134 to the GPU memory 152.

For example, in a case where the frame is divided into four strip images similarly to the case of the frame image FP illustrated in FIG. 6, the output timing is delayed by at least three strip images with respect to the input timing. Moreover, in order to absorb fluctuation (jitter) due to software processing, the output timing may be delayed by four strip images with respect to the input timing.

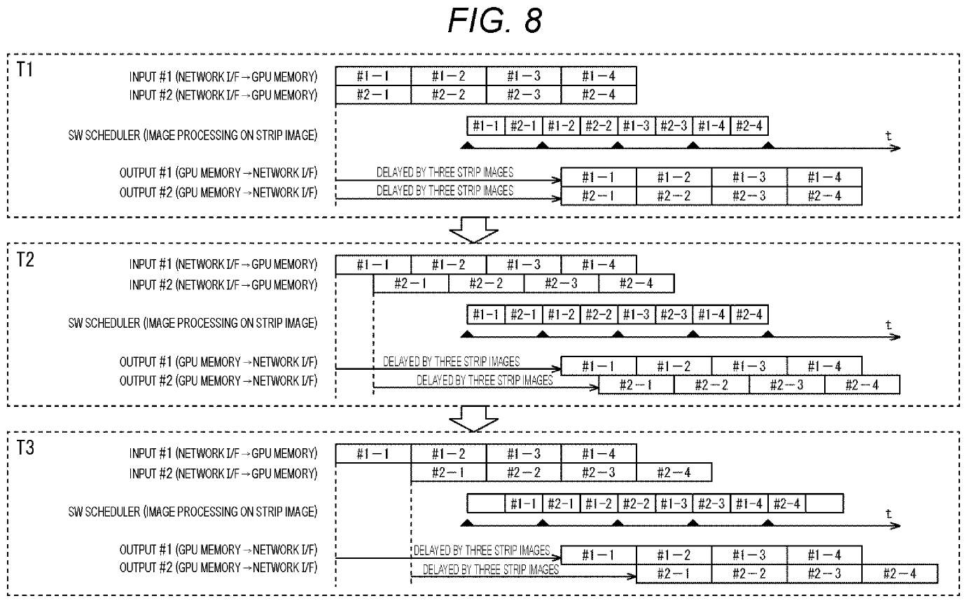

Here, a specific example of the processing timing of the image processing by the image processing server 110 is described with reference to FIG. 8.

In the example of FIG. 8, image processing is executed on data (input #1) input from an IP converter #1 on the image transmission device 10 side, and image processing is executed on data (input #2) input from an IP converter #2 on the image transmission device 10 side.

Furthermore, the data of the input #1 subjected to the image processing is output as output #1 to the IP converter #1 on the image reception device 20 side, and the data of the input #2 subjected to the image processing is output as output #2 to the IP converter #2 on the image reception device 20 side.

FIG. 8 illustrates a temporal flow of transmission of data to the GPU memory 152 and image processing on the GPU memory 152 in one predetermined frame at each of times T1, T2, and T3. At each time, data of one frame of each of the inputs #1 and #2 is divided into four strip images, and the strip images are indicated by rectangles in which #1 or #2 indicating the input or output is assigned and then branch numbers of 1 to 4 are assigned after the #1 or #2.

Note that in the example of FIG. 8, it is assumed that the frequency of the vertical synchronization signal of the medical image related to the IP converter #2 is lower than the frequency of the vertical synchronization signal of the medical image related to the IP converter #1, and the input #2 is delayed with respect to the input #1 while the time from time T1 to time T3 elapses.

(Time T1)

At time T1, the data of the input #1 and the data of the input #2 are transferred and deployed from the network I/F 134 to the GPU memory 152 at the same timing.

The SW scheduler executes image processing on the data of the input #1 and the data of the input #2, which are deployed on the GPU memory 152 in units of strip images at each processing timing based on the interrupt signal indicated by a triangle on a time axis t.

For example, at a first processing timing, the image processing is executed on a strip image #1-1 and a strip image #2-1, which are deployed on the GPU memory 152, by the SW scheduler. At a second processing timing, the image processing is executed on a strip image #1-2 and a strip image #2-2, which are deployed on the GPU memory 152, by the SW scheduler.

As described above, on the GPU memory 152, the image processing is sequentially and collectively executed on the inputs containing the data of the strip image. Thus, when image processing required by a plurality of applications is executed, it is possible to reduce overhead related to an execution request to the GPU card 135 and synchronization processing as compared with a case where the image processing is executed at each timing. As a result, low-latency image processing can be implemented.

The data subjected to the image processing are respectively read as the output #1 and the output #2 at the same timing from the GPU memory 152 to the network I/F 134. At this time, the output #1 and the output #2 are delayed by three strip images with respect to each of the input #1 and the input #2, respectively, and are read from the GPU memory 152.

(Time T2)

At time T2, the data of the input #2 is transferred and deployed from the network I/F 134 to the GPU memory 152 at a timing delayed with respect to the data of the input #1.

Here, even when the data of the input #2 is delayed with respect to the data of the input #1, the strip image #1-1 and the strip image #2-1 are aligned on the GPU memory 152 at the first processing timing. Therefore, at the first processing timing, the image processing is executed on the strip image #1-1 and the strip image #2-1, which are deployed on the GPU memory 152, by the SW scheduler. At the second processing timing, the image processing is executed on a strip image #1-2 and a strip image #2-2, which are deployed on the GPU memory 152, by the SW scheduler.

The data subjected to the image processing are respectively read as the output #1 and the output #2 from the GPU memory 152 to the network I/F 134 at a timing at which the output #2 is delayed with respect to the output #1. At this time, the output #1 and the output #2 are delayed by three strip images with respect to each of the input #1 and the input #2, respectively, and are read from the GPU memory 152.

(Time T3)

At time T3, the data of the input #2 is transferred and deployed from the network I/F 134 to the GPU memory 152 at a timing delayed by one strip image with respect to the data of the input #1.

Here, since the data of the input #2 is delayed by one strip image with respect to the data of the input #1, the strip image #1-1 is aligned on the GPU memory 152 at the first processing timing, but the strip image #2-1 is not aligned. Therefore, at the first processing timing, the image processing is executed on only the strip image #1-1 deployed on the GPU memory 152 by the SW scheduler. That is, the image processing on the data of the input #2 is skipped by one strip image. At this time, the processing amount in the GPU card 135 is reduced. Thereafter, at the second processing timing, the image processing is executed on the strip image #2-1 and the strip image #1-2, which are deployed on the GPU memory 152, by the SW scheduler.

The data subjected to the image processing are respectively read as the output #1 and the output #2 from the GPU memory 152 to the network I/F 134 at a timing at which the output #2 is delayed by one strip image with respect to the output #1. At this time, the output #1 and the output #2 are delayed by three strip images with respect to each of the input #1 and the input #2, respectively, and are read from the GPU memory 152.

As described above, the output #1 and the output #2 are respectively delayed by three strip images with respect to the input #1 and the input #2. In a case where the input #1 and the input #2 are completely synchronized and input, the output #1 and the output #2 may be delayed by two strip images.

However, in a case where the input #1 and the input #2 are input asynchronously, as described above, image processing on data input late is skipped, and thus a delay corresponding to three strip image is required. Accordingly, image processing is sequentially and collectively executed even on data input asynchronously in units of strip images, and thus missing of frames can be prevented. Furthermore, as described above, in order to absorb fluctuation (jitter) due to software processing and further increase stability, the output is delayed by four strip images with respect to the input.

### Modification Example

As described above, the image processing on the strip image that is not developed on the GPU memory 152 is skipped, but for example, the network I/F 134 may notify the SW scheduler of a transfer state of the data to the GPU memory 152. In this case, when notification that the transfer of the data corresponding to one strip image from the network I/F 134 has not been completed is provided to the SW scheduler, the image processing may be skipped.

Note that whether or not to skip image processing may be determined on the basis of the type of image transmission device 10 that inputs a medical image, such as a medical imaging device.

Furthermore, in a case where incompleteness such as mixture of strip images of different frames occurs in data subjected to the image processing, the data being transferred from the GPU memory 152 to the network I/F 134, correction processing on synchronization deviation or the like may be performed by the network I/F 134.

Moreover, in a case where the processing amount of the image processing executed on the strip images of each medical image at one processing timing is a processing amount that cannot be processed at the processing timing, an alert to the user may be output.

As described above, the strip image is deployed on the GPU memory 152, but the strip image may be deployed on the main memory 132, and then image processing may be executed or skipped according to a state of data transfer to the main memory 132.

<5. Configuration Example of Computer>

A series of processing described above can be executed by hardware or can be executed by software. In a case of executing the series of processing by the software, a program configuring the software is installed from a program recording medium into a computer built into dedicated hardware, a general-purpose personal computer, or the like.

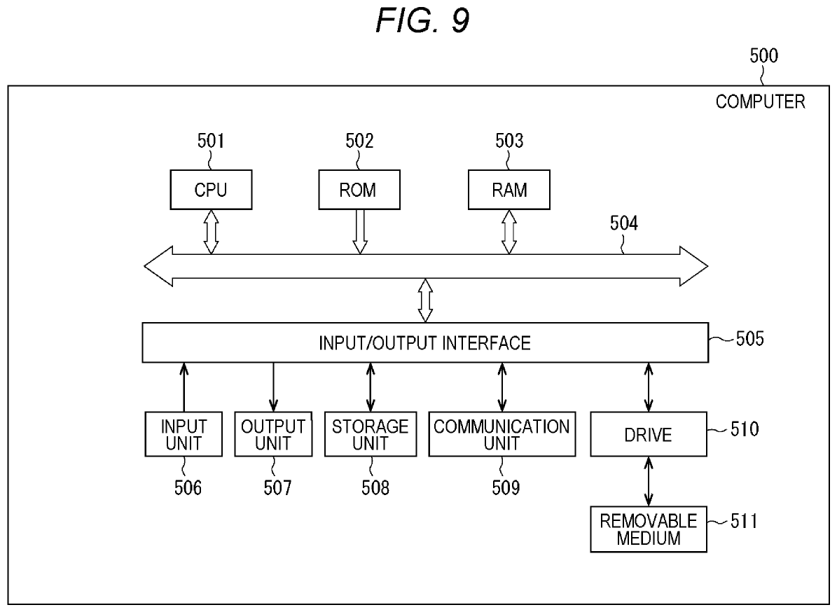

FIG. 9 is a block diagram illustrating a configuration example of the hardware of the computer, which executes the above-described series of processing by the program.

The medical image processing system 100 (image processing server 110) to which the technology according to the present disclosure can be applied is implemented by the computer having the configuration illustrated in FIG. 9.

A CPU 501, a read only memory (ROM) 502, and a random access memory (RAM) 503 are mutually connected via a bus 504.

An input/output interface 505 is further connected to the bus 504. An input unit 506 including a keyboard and a mouse, and an output unit 507 including a display and a speaker are connected to the input/output interface 505. Furthermore, a storage unit 508 including a hard disk and a nonvolatile memory, a communication unit 509 including a network interface and a drive 510 that drives a removable medium 511 are connected to the input/output interface 505.

In the computer configured as described above, for example, the CPU 501 loads a program stored in the storage unit 508 into the RAM 503 via the input/output interface 505 and the bus 504 and executes the program to perform the above-described series of processing.

For example, the program to be executed by the CPU 501 is recorded in the removable medium 511 or provided via a wired or wireless transmission medium such as a local area network, the Internet, or a digital broadcast, and installed in the storage unit 508.

Note that the program to be executed by the computer may be a program with which the processing is performed in time series in the order described herein, or may be a program with which the processing is performed in parallel or at necessary timing such as a timing at which a call is made.

The embodiment of the present disclosure is not limited to the above-described embodiment, and various modifications can be made without departing from the gist of the present disclosure.

Furthermore, the effects described herein are merely examples and are not limited, and other effects may be provided.

Moreover, the present disclosure can also have the following configurations.

(1)

A medical image processing system including
an image processing unit configured to execute, at each predetermined processing timing, image processing on each of a plurality of medical images input asynchronously in units of strip images obtained by dividing each of the medical images into a plurality of pieces.

(2)

The medical image processing system according to (1),
in which the processing timing is a timing obtained by multiplying a frequency higher than that of a vertical synchronization signal of any of the medical images.

(3)

The medical image processing system according to (2),
in which a multiplication number is a division number of the strip image.

(4)

The medical image processing system according to any one of (1) to (3),
in which the strip image is an image obtained by dividing the medical image into a plurality of pieces in a horizontal direction.

(5)

The medical image processing system according to any one of (1) to (4),
in which the image processing unit executes, at the processing timing, the image processing of each of the medical images on the strip image for which data deployment on a memory is completed.

(6)

The medical image processing system according to (5),
in which in the image processing on each of the medical images, the image processing unit skips, at the processing timing, the image processing on the strip image for which the data deployment on the memory is not completed.

(7)

The medical image processing system according to (6),
in which the data is deployed to different regions on the memory for each strip image.

(8)

The medical image processing system according to (6) or (7), further including an image processing server including the image processing unit,
in which the image processing server delays a timing to output the data subjected to the image processing from the memory in accordance with a division number of the strip image with respect to a timing to input the data not subjected to the image processing to the memory.

(9)

The medical image processing system according to (8),
in which the image processing server includes a network I/F that notifies the image processing unit of a state of transfer of the data to the memory.

(10)

The medical image processing system according to (9),
in which the image processing server includes a direct memory access (DMA) controller configured to execute direct transfer of the data not subjected to the image processing from the network I/F to the GPU memory and direct transfer of the data subjected to the image processing from the GPU memory to the network I/F.

(11)

A medical image processing method including
causing a medical image processing system to execute, at each predetermined processing timing, image processing on each of a plurality of medical images input asynchronously in units of strip images obtained by dividing each of the medical images into a plurality of pieces.

(12)

A program causing a computer to execute, at each predetermined processing timing, image processing on each of a plurality of medical images input asynchronously in units of strip images obtained by dividing each of the medical images into a plurality of pieces.

### REFERENCE SIGNS LIST

1 Medical image processing system
10 Image transmission device
11 IP converter
20 Image reception device
21 IP converter
30 IP switch
40 Manipulation terminal
50 Control server
100 Medical image processing system
110 Image processing server
131 CPU
132 Main memory
133 Bus
134 Network I/F
135 GPU card
151 Processor
152 Memory
211 Image processing unit
212 Application group
213 Interrupt signal generation unit

1. A medical image processing system comprising
an image processing unit configured to execute, at each predetermined processing timing, image processing on each of a plurality of medical images input asynchronously in units of strip images obtained by dividing each of the medical images into a plurality of pieces.

2. The medical image processing system according to claim 1,
wherein the processing timing is a timing obtained by multiplying a frequency higher than that of a vertical synchronization signal of any of the medical images.

3. The medical image processing system according to claim 2,
wherein a multiplication number is a division number of the strip image.

4. The medical image processing system according to claim 1,
wherein the strip image is an image obtained by dividing the medical image into a plurality of pieces in a horizontal direction.

5. The medical image processing system according to claim 1,
wherein the image processing unit executes, at the processing timing, the image processing of each of the medical images on the strip image for which data deployment on a memory is completed.

6. The medical image processing system according to claim 5,
wherein in the image processing on each of the medical images, the image processing unit skips, at the processing timing, the image processing on the strip image for which the data deployment on the memory is not completed.

7. The medical image processing system according to claim 6,
wherein the data is deployed to different regions on the memory for each strip image.

8. The medical image processing system according to claim 6, further comprising an image processing server including the image processing unit,
wherein the image processing server delays a timing to output the data subjected to the image processing from the memory in accordance with a division number of the strip image with respect to a timing to input the data not subjected to the image processing to the memory.

9. The medical image processing system according to claim 8,
wherein the image processing server includes a network I/F that notifies the image processing unit of a state of transfer of the data to the memory.

10. The medical image processing system according to claim 9,
wherein the image processing server includes a direct memory access (DMA) controller configured to execute direct transfer of the data not subjected to the image processing from the network I/F to the GPU memory and direct transfer of the data subjected to the image processing from the memory to the network I/F.

11. A medical image processing method comprising causing a medical image processing system to execute, at each predetermined processing timing, image processing on each of a plurality of medical images input asynchronously in units of strip images obtained by dividing each of the medical images into a plurality of pieces.

12. A program causing a computer to execute, at each predetermined processing timing, image processing on each of a plurality of medical images input asynchronously in units of strip images obtained by dividing each of the medical images into a plurality of pieces.
